(19) United States
(12) Patent Application Publication
(10) Pub. No.: US 2009/0264878 A1
(43) Pub. Date: Oct. 22, 2009

(54) DEVICES AND METHODS FOR ABLATING AND REMOVING A TISSUE MASS

(75) Inventors: Yuval CARMEL, Rockville, MD (US); Robert A. Van Wyk, Largo, FL (US); Anatoly Shkvarunets, Rockville, MD (US)

Correspondence Address: SMITH PATENT CONSULTING, LLC, 3307 DUKE STREET, ALEXANDRIA, VA, 22314, US

(73) Assignee: Electro Medical Associates, LLC, Bethesda, MD (US)

(21) Appl. No.: 12/426,398

(22) Filed: Apr. 20, 2009

Related U.S. Application Data
(60) Provisional application No. 61/124,971, filed on Apr. 21, 2008.
(60) Provisional application No. 61/139,979, filed on Dec. 22, 2008.

(51) Int. Cl. A61B 18/18; A61B 18/14

(52) U.S. Cl. 606/33; 606/42; 606/41

(57) ABSTRACT

Disclosed herein are high efficiency surgical devices and methods of using same using radio frequency (RF) electrical power to destroy, vaporize and remove soft tissues, such as tumors, both malignant and benign, from within a target surgical site. In one particularly preferred embodiment, the electrosurgical device employs a combination of rotary and translational motion to incrementally vaporize a calculated volume of tissue. According to the principles of this invention, the electrosurgical devices can be used with externally supplied conductive or non-conductive irrigants, whether liquid, gas, or a combination thereof, as well as without externally supplied liquids, a mode of operation often referred to as “dry field” environment. The electrosurgical devices may further optionally include aspiration components to permit removal of vaporization by-products.

### PRIORITY

This application claims the benefit of U.S. Provisional Application Ser. Nos. 61/124,971, filed Apr. 21, 2008, and 61/139,979, filed Dec. 22, 2008, the entire contents of which are hereby incorporated by reference herein.

### FIELD OF THE INVENTION

The present invention relates generally to the field of electrosurgery, and more particularly, to high efficiency surgical devices and methods which use radio frequency (RF) electrical power to ablate, denature, vaporize and remove all or part of a tissue mass, with or without the use of externally supplied liquids or gases, as well as to thermally treat (i.e., cauterize, coagulate, form lesions in) any remaining tissue.

### BACKGROUND OF THE INVENTION

It is well known in the prior art to use high frequency current in electrosurgical devices to perform many divergent surgical procedures. Electrosurgical procedures are advantageous since they generally reduce patient bleeding and trauma. The devices used are electrically energized, typically using an RF generator operating at a frequency between 100 kHz to over 6 MHz. The instruments may also be energized by microwave or millimeter wave generators operating at a frequency range of 500 MHz to over 30 GHz. Due to their ability to provide beneficial outcomes with reduced patient pain and recuperation time, electrosurgical devices have recently gained significant popularity. In common terminology and as used herein, the term “electrode” can refer to one or more components of an electrosurgical device (such as an “active electrode” or a “return electrode”) or to the entire device, as in an “ablator electrode” or “cutting electrode”. Electrosurgical devices may also be referred to as electrosurgical “probes” or “instruments”.

Electrosurgery probes may be used for vaporization of tissue or for thermal modification of the tissue, such as lesion formation. Vaporization of tissue occurs when the local current density at the active electrode is sufficiently high to cause localized boiling of fluid at the active electrode, and arcing within the bubbles formed. When the current density is insufficient to cause boiling, the tissue in proximity to the active electrode is exposed to both current and high-temperature liquid, the temperature of the tissue and liquid being affected by the current density at the active electrode, and the flow of fluid in proximity to the electrode. The current density is determined by the probe design and by the voltage applied to the probe. A given probe, therefore, may function as either a vaporizing probe or a thermal treatment probe depending on the choice of voltage applied to the probe. Lower voltages will cause a probe to operate in the thermal treatment mode rather than in the vaporizing mode that is possible if higher voltage is applied. In some cases, an externally supplied liquid (also referred to as an “irrigant”, either electrically conductive or non-conductive) is used. In other electrosurgical procedures, the devices rely only on locally available bodily fluids, without requiring an external source of fluids. Procedures performed this way are sometimes referred to as performed in dry-field, or semi-dry. Yet other electrosurgical instruments may be equipped with irrigation, aspiration or both.

Many types of electrosurgical instruments are currently in use, and can be divided into two general categories: monopolar devices and bipolar devices. In the context of monopolar electrosurgical devices, the RF current generally flows from an exposed active electrode, through the patient's body, to a passive, return current electrode that is externally attached to a suitable location on the patient body. In this manner, the large volume of the patient's body becomes part of the return current circuit. In the context of bipolar electrosurgical devices, both the active and the return current electrodes are exposed, and are typically positioned in close proximity to each other, more frequently mounted on the same instrument. The RF current flows from the active electrode to the return electrode through the nearby small volume of tissue and conductive fluids.

Electrosurgical devices that cut or vaporize tissue rely on generation of sparks in the vicinity of the active electrodes to vaporize the tissue. Sparking is also often referred to as arcing within bubbles or alternatively as plasma. The geometry, shape and material of the electrosurgical device, as well as the particular tissue properties, can greatly affect the device's performance, safety and reliability. Inefficiently designed devices require substantially higher power levels than those with more efficient designs.

Recently, specialized electrosurgical probes called “ablators” have been developed for the bulk vaporization of tissue. Rather than cutting out discrete pieces of tissue, volumes of tissue are vaporized and removed from the site as gasses and vaporization byproducts. Commercial examples of such instruments include ArthroWands manufactured by Arthrocare (Sunnyvale, Calif.), VAPR electrodes manufactured by Mitek Products Division of Johnson & Johnson (Westwood, Mass.), Stryker Corporation (Kalamazoo, Mich.) and Smith and Nephew Endoscopy (Andover, Mass.). These ablators differ from conventional arthroscopic electrosurgical probes in that they are designed for the bulk removal of tissue by vaporization in a conductive liquid environment rather than only for cutting of tissue or for coagulation of bleeding vessels.

During ablation, the fluids within the target tissue are vaporized. Because volumes of tissue are vaporized rather than discretely cut out and removed from the surgical site, the power requirements for ablator electrodes are generally higher than those of other arthroscopic electrosurgical electrodes. The geometry and design of the electrode and the characteristics of the RF power supplied to the electrode greatly affect the efficiency and power required for ablation (bulk vaporization) of tissue. Electrodes with inefficient designs will require higher power levels than those with efficient designs in order to achieve the desired medical effect. The physics of the ablation (vaporization) process and the effect of ablator device construction features on ablation efficiency are extensively discussed in U.S. Pat. Nos. 7,166,103, 6,921,399, 6,921,398, 6,796,982 and 6,840,937, the contents of which are hereby incorporated by reference herein in their entirety.

The present inventors previously discovered that the efficiency of an electrosurgical probe could be dramatically increased by the additional of one or more electrically conducting (metallic or other) active elements which are not connected directly to any part of the external power supply and having so called “floating” potential (voltage). These active elements are referred to herein and elsewhere as “floating electrodes” to reflect the electrical potential of such elements.

The additional conducting active elements with floating potential may contact the surrounding bodily fluid, conducting fluid/liquid and/or tissue. When properly designed according to the principles of this invention, the presence of these additional conducting floating elements favorably modifies the distribution of the energy in its vicinity and in the vicinity of the active electrode(s). The active element is electrically “floating”, in the sense that it is not directly connected to the external RF energy source. The electrical potential of the floating active element depends on the size and position of the element, the tissue type and properties, the presence or absence of bodily fluids or externally supplied fluid, and the RF voltage used. This “floating” element(s) is mounted in such a way that one portion of the element(s) is in close proximity to the probe tip, in the region of high potential. Another portion of the floating element(s) is placed further away in a region of otherwise low potential.

The floating element increases the concentration of high power density in the vicinity of the active region, and results in more focused and efficient liquid heating, steam bubble and layer formation and bubble trapping in this region. This allows high efficiency operation, which in turn increases patient safety by allowing the surgeon to substantially decrease the applied to RF power. The physics, principles of operation, and construction of electrosurgical devices incorporating floating potential active elements are fully described in co-pending U.S. application Ser. Nos. 10/911,309, filed Aug. 4, 2004 and 11/136,514, filed May 25, 2005, both of which are pending allowance, the entire contents of which are hereby incorporated by reference herein in their entirety.

Recent improvements in tumor detection methods and systems have allowed for the identification and location of small tumors, frequently as small as one millimeter. The use of RF to destroy (i.e., kill, denaturize) tumors through thermal treatment is well known. Many patients, however, prefer the removal of a tumor rather than merely thermally killing the tumor and leaving its remnants in place (in situ). However, for tumors within tissue or an organ, rather than on the surface of an organ, removal can be difficult or impossible.

Electrosurgical instruments that are designed to thermally kill, or denature, soft tissue are also known in the prior art and are sometimes referred to as Radiofrequency Ablation (RFA) instruments. RFA instruments, both monopolar and bipolar, are gaining wider acceptance. This approach involves substantially no sparking or arcing. RFA is a minimally invasive procedure used to destroy lesions in which the deposition of radiofrequency energy produces thermal injury to the target tissue. RFA can be performed using an open, percutaneous, or laparoscopic technique. An electrical current from exposed areas of the electrosurgical instrument is delivered to the tissue, it generates heat that is high enough to create lesions and kill the lesion cells (thermal treatment). Heating of soft tissue above 50° C. causes numerous changes at the cellular level including denaturization of protein and loss of intracellular fluids, a process sometimes called desiccation.

RFA instruments can be used in percutaneous, laparoscopic or open procedures. Commercial examples of RFA instruments are those marketed by RITA Medical Systems, Inc. (Mountain View, Calif.), AngioDynamics (Queensbury, N.Y.), Boston Scientific (Boston, Mass.), RF Medical Inc. (Fremont Calif.), Medtronic (Minneapolis, Minn.) and Covidien (previously Valleylab, Boulder Colo.). However, while RFA instruments of the prior art are useful in destroying tumors, both benign and malignant, in various organs such as liver, it is important to note that in most cases the tissue is neither evaporated nor removed from the body, but simply denatured and left in place. Over time, the treated denatured tissue is gradually absorbed and naturally removed from the body in a process that may take up to few months. In that it is possible for the residual denatured tissue to regain its oncogenic potential, proliferate and/or metastasize, this is perceived by many to be an undesirable end result.

Thus, even though the benefits of RFA instruments of the prior art are well recognized, these devices and procedures suffer from significant deficiencies. For example, current RFA procedures are time consuming, lasting in some cases up to several hours. Also, they may produce non-uniform heating. The thermal effects produced by RFA cause a decrease in the ability of soft tissue to conduct electrical current. This causes a rise in the level of resistance to current flow, also referred to as the tissue impedance. This effect may prematurely decrease the current flow. It also limits the transfer of energy to tissue in close vicinity to the electrode and may result in a “kill volume” that is non-uniform. There may be regions in which malignant or other unhealthy or undesired tissue is either untreated or under treated. Extending the duration of the procedure or increasing the RF current will not help to alleviate this problem.

In addition, in the context of conventional RFA procedure, treated tissue is not removed from the body, leaving untreated or under-treated regions of undesirable tissue at the site. Furthermore, prior art RFA devices cannot be repositioned during the procedure. Thus, it is difficult to match the treatment region to the size and shape of the mass to be treated.

Finally, patient may receive internal or external burns due to the high currents used. In some cases the current needed to achieve the desired medical effect during the procedure is so high that up to four return electrodes (sometimes referred to as dispersive electrode or grounding pad) must be attached to the patient. Often it is very difficult to find suitable locations on the patient body to locate multiple electrodes, especially for elderly people, people with skin problems, excessive hair or children. This limits the usefulness of the technique.

Accordingly, there is a need for a minimally invasive RF device which will overcome the significant deficiencies described above, and is able to effectively and safely volumetrically vaporize and remove a lesion or tumor, even small tumors on the order of 1 cm or less, within tissue and to thermally treat the surrounding tissue. The present inventors submit that the instant invention meets this need.

### SUMMARY OF THE INVENTION

It is accordingly an object of the present invention to provide a minimally invasive electrosurgical device which capable of volumetrically vaporizing a tumor or other tissue within a tissue mass from the patient body.

It is another object of the present invention to provide a minimally invasive electrosurgical device capable of removing the vaporization by-products via aspiration, suction or other means.

It is a further object of the present invention to provide a minimally invasive electrosurgical device capable of thermally treating neighboring, remaining and/or surrounding tissue.

It is yet a further object of the present invention to provide an electrosurgical device wherein both the axial and radial position of the active and/or floating electrodes can be controlled and adjusted, either manually or automatically, during the procedure.

It is yet a further object of the present invention to provide an electrosurgical device that employs a combination of rotary and translational motion to incrementally vaporize a calculated volume of tissue, for example, wherein the active and/or floating electrodes can be rotated, or oscillated around the shaft of the instrument during the procedure, thus allowing for incremental volumetric evaporation of large cavities.

It is yet a further object of the present invention to provide an electrosurgical device that is compatible with most general purpose RF generators as well as stabilizing mechanisms and robotic surgery systems.

It is yet a further object of the present invention to provide an electrosurgical device that can easily penetrate mobile lesions or tumors, in organs like liver, kidney, lung, breast, bone, and brain as well as others.

It is yet a further object of the present invention to provide an electrosurgical device with integrated handpiece with cable, mechanisms to move the active and floating electrodes, and a power source for electrode movement, all disposable.

It is yet a further object of the present invention to provide a high efficiency disposable electrosurgical device.

It is yet a further object of the present invention to provide an electrosurgical device capable of operating in electrically conductive and non-conductive fluid environments, as well as in dry or semi dry environment (bodily fluids), gaseous environment or a combination of gas and liquid.

It is yet a further object of the present invention to provide an electrosurgical device that can penetrate the body by using electrical, mechanical means, or via one or more trocars, introducers, resectoscopes, canulas or other introducing devices in order to perform the procedure at the precise target location within the body.

It is yet a further object of the present invention to provide an electrosurgical, image-guided device that is compatible, and can be used in conjunction with a real-time or off-line imaging system.

It is yet a further object of the present invention to provide an electrosurgical device having enhanced safety and shortened procedure time.

Accordingly, an electrosurgical device produced in accordance with the principles of the present invention can volumetrically vaporize and remove undesirable tissue from organs such as the liver, brain, kidney, lung, bone, breast within the body. The working element (or as it is sometimes called the “active element” or “active electrode”) is energized (via RF power applied) and brought into contact with tissue to be vaporized. This leads to volumetric evaporation of the target tissue. In a first aspect, the device may be manually repositioned to effect vaporization of a volume. In a second aspect, the device is provided with means within its handle portion to translate the effecting portion radially and axially while energized so as to incrementally vaporize a large volume of tissue.

In a first embodiment, configured for the incremental vaporization and thermal treatment of small tumors, the present invention provides an electrosurgical device having a proximal portion forming a handle, and a distal portion having at its distal end at least one active electrode and at least one floating electrode, the distal end being sharpened so as to allow it to penetrate tissue, and the at least one active electrode being positioned slightly proximal to the sharpened distal end. The active electrode is connected via cabling and means within the elongated proximal portion to an electrosurgical generator. At least a portion of the distal-most portion of at least one floating electrode is in close proximity to at least one active electrode. In one preferred embodiment, the active electrode has a plurality of grooves formed in its ablating surface, the grooves being of a depth and width for maximal retention of bubbles within the grooves. In other embodiments, the active surface may comprise a continuous or discontinuous array of raised and recessed portions having any number of possible geometries, including, for example, linear, curvilinear, polygonal, rectangular, circumferential and others, or any combination thereof. The active electrode may optionally be provided, at least in part, with a layer of electrical insulation fabricated from a suitable dielectric material.

The floating electrode may surround the active electrode and may be separated from it by a dielectric member. The floating electrode intensifies the electric field in proximity to the active electrode and aids bubble retention when the probe is used to vaporize tissue. In a preferred embodiment, the device has irrigation supplied to the probe tip, and a means for aspirating vaporization byproducts and fluid from the site. Irrigation and aspiration may be controlled by means within the handle, or by an external means. The irrigation means and aspiration means may use a common lumen in the elongated distal portion. The active and floating electrodes may be used for vaporizing tissue by applying sufficient voltage for bubble formation and arcing, or may be used for thermal treatment of tissue by applying lower voltages.

The present invention further provides methods for removing and treating a tumor within a tissue mass. In one embodiment, the device of the present invention is connected to a suitable RF generator, to an external vacuum source, and to an irrigant source. Under image guidance, the elongated distal portion of the portion of the device is inserted into the tissue, the distal end of the portion being located within the tumor. The generator is set to a first power level. Irrigant is supplied to the site through the device distal tip, the pressure of the supplied irrigant causing a cavity to be formed within the tumor, the distal tip of the instrument being within the cavity. The active electrode is positioned adjacent to tissue and the generator is activated for a brief period of time, generally for seconds or minutes. During activation, tissue in close proximity to the active electrode is vaporized. Subsequently, debris and fluid are aspirated from the site. The active electrode is manually repositioned axially and/or by rotation about the device axis to a second location and the process repeated. The steps are repeated until the tumor has been completely vaporized and aspirated from the site. Optionally, after the tumor has been removed, the RF generator may be set to a second power level and, while irrigant is supplied to the site, activated. The second power level is such that arcing and vaporization of tissue do not occur, but rather that the irrigant and adjacent tissue are heated so as to thermally treat tissue surrounding the site. The devices according to the principles of this invention can be designed to penetrate the body using mechanical, electrical or other means, or via one or more trocars, introducers, resectoscopes or cannulas.

In a second embodiment, the present invention provides an electrosurgical device adapted for the incremental vaporization of larger tumors wherein volumes can be vaporized and, optionally, the site thermally treated without manual repositioning of the device. In many cases the volume and size of the tumor or lesion to be removed is significant and it may be located deep inside the organ. Manual manipulation of the probe is frequently restricted and the required tissue removal cannot be achieved. More over, in many cases off-axis motion of the shaft of the probe is practically impossible, because of the presence of bones in the body that may restrict motion, or because the presence of sensitive vital organs or blood vessels. In contrast, the longitudinal motion and rotation around axis is unrestricted.

Devices of this second embodiment are preferably equipped with at least one active electrode capable of volumetric evaporation of tissue. The active electrode according to the principles of this invention may be rotated around the shaft of the instrument during the procedure by means within the handle portion, thus allowing for volumetric evaporation of large cavities. The rotational motion can be symmetric or non symmetric, uni or bi directional and may also include oscillation, vibration and translation, or a combination thereof. In one of the preferred embodiments the radial position of the tip is adjusted by the rotational motion of a control rod. The probe according to the principles of this invention can be of multi electrode design including one or more floating electrodes, and can be equipped with either irrigation or aspiration (suction), or both. The irrigation and aspiration can be integral, or separate from the instrument.

In a preferred embodiment, the electrosurgical device is an RF instrument having a proximal portion forming a handle and an elongated, closed-end, cannulated distal portion provided with a sharpened point distal end for penetrating the target tissue, for example the exterior membrane/epithelial layer of a tumor mass. Alternatively, penetration of the device into the tissue may be achieved using electrical power or other means. The cannulated distal portion has at its distal end an elongated slot. The proximal end of the cannulated portion has a threaded portion assembled thereto. An elongated active electrode is slidably positioned within the lumen of the elongated cannulated portion such that when the proximal end of the active electrode is displaced axially by a threaded element positioned within the threaded portion of the cannulated portion and rotated relatively thereto, the distal portion of the active electrode is deformed so as to form a protruding loop through the distal slot in the cannulated member. In use, the distal end of the cannulated member is positioned slightly past the distal extremity of a mass to be removed. A motor within the handle rotates the cannulated member such that the threaded element screws into the threaded end of the cannulated portion. Each rotation of the cannulated member causes the threaded member to advance a predetermined distance which, in turn, causes the distal portion of the active electrode to be deformed a predetermined distance from the distal slot in the cannulated member. RF energy supplied to the active electrode vaporizes tissue with which it comes in contact. The position of the distal end is maintained by the user, with the cannulated member rotating about its axis. The distal portion of the electrode is increasingly deformed from the distal slot by the threaded member so that each rotation of the cannulated member and electrode increases the diameter of the void formed by vaporization of tissue contacting the electrode. The rate at which the electrode advances into the tissue is determined by the pitch of the thread of the threaded member and threaded portion of the cannulated member. At a location within the tissue mass, vaporization is accomplished in an incremental manner with each sweep of the active electrode through the location, the increment being determined by the size of the distal slot and the pitch of the threaded portions. Tissue is incrementally vaporized until the threaded member reaches a predetermined position within the threaded portion of the cannulated member, whereupon the device is de-energized. Rotation of the cannulated member is then reversed so that the threaded element moves proximally and the active electrode proximal end is drawn proximally so as to bring the deformed distal portion of the active element back into the cannulated member. Alternatively, the threaded member may be disengaged from the threaded portion of the cannulated member so as to allow the electrode to be withdrawn proximally into the cannulated member.

In a preferred embodiment, gasses and other by-products resulting from the vaporization of tissue may be aspirated from the site through the cannulated member. In a more preferred embodiment, the elongated cannulated portion may be formed from one or more dielectric materials chosen from the group including but not limited to alumina, zirconia and polymeric materials including composite materials. In other embodiments, the elongated cannulated portion may be provided with an outer surface covered with a dielectric coating.

As noted above, it is an object of the present invention to provide an electrosurgical device wherein both the axial and radial position of the active and/or floating electrodes can be controlled and adjusted, either manually or automatically, during the procedure. To that end, the present invention contemplates not only automated coordination between rotational movement at the proximal end (e.g., via manipulation of the handle or proximal housing) and axial movement at the distal end (e.g., projection or protrusion of at least a distal portion of the active electrode into the predetermined surgical site), but further contemplates assisted support for the device as a whole and/or control of all or part of the operation thereof. For example, the present invention contemplates mounting an electrosurgical device of the present invention to external support hardware or other means for stabilizing the device in operation. The present invention also contemplates coupling an electrosurgical device of the present invention to a robotic surgery system that may provide remote and/or automated manipulation all or part of its operation.

These and other objects and features of the invention will become more fully apparent when the following detailed description is read in conjunction with the accompanying figures and/or examples. However, it is to be understood that both the foregoing summary of the invention and the following detailed description are of a preferred embodiment and not restrictive of the invention or other alternate embodiments of the invention. In particular, while the invention is described herein with reference to a number of specific embodiments, it will be appreciated that the description is illustrative of the invention and is not constructed as limiting of the invention. Various modifications and applications may occur to those who are skilled in the art, without departing from the spirit and the scope of the invention, as described by the appended claims. Likewise, other objects, features, benefits and advantages of the present invention will be apparent from this summary and certain embodiments described below, and will be readily apparent to those skilled in the art having knowledge of electrode design. Such objects, features, benefits and advantages will be apparent from the above in conjunction with the accompanying examples, data, figures and all reasonable inferences to be drawn there-from, alone or with consideration of the references incorporated herein.

In addition, it will be understood by those skilled in the art that one or more aspects of this invention can meet certain of the above objectives, while one or more other aspects can meet certain other objectives. Each objective may not apply equally, in all its respects, to every aspect of this invention. As such, the preceding objects should be viewed in the alternative with respect to any one aspect of this invention.

### BRIEF DESCRIPTION OF THE FIGURES

Various aspects and applications of the present invention will become apparent to the skilled artisan upon consideration of the brief description of the figures and the detailed description of the present invention and its preferred embodiments which follows:

FIG. 1 is a schematic view of an electrosurgical system constructed in accordance with the principles of this invention.

FIG. 2 is a schematic view of a probe constructed in accordance with the principles of this invention.

FIG. 3 is a perspective view of an electrosurgical probe constructed in accordance with the principles of this invention.

FIG. 4 is an expanded plan view of the distal portion of the probe of FIG. 3.

FIG. 5 is a side elevational view of the objects of FIG. 4.

FIG. 6 is a perspective view of the objects of FIG. 4.

FIG. 7 depicts the distal portion of the probe of FIG. 3 during use.

FIG. 8 is an expanded view of the distal region of FIG. 7.

FIG. 9 depicts the probe of FIG. 2 as positioned by an imaging system.

FIG. 10 is a schematic representation of an alternate embodiment for vaporizing larger tumors as positioned by an imaging system.

FIG. 11A is a schematic representation of the object of FIG. 10 at the start of a tumor removal cycle. FIG. 11B is a schematic representation of the object of FIG. 10 at the completion of a tumor removal cycle.

FIG. 12A depicts a perspective exploded view of the inner assembly of the device of FIG. 10. FIG. 12B is an expanded perspective view of the medial portion of the objects of FIG. 12A

FIG. 13 is a side elevational view of the objects of FIG. 12A.

FIG. 14A is a perspective view of the assembled objects of FIG. 12A. FIG. 14B is an expanded perspective view of the medial portion of the objects of FIG. 14A

FIG. 15 is a side elevational view of the objects of FIG. 14A.

FIG. 16 depicts a perspective view of the outer assembly of the device of FIG. 10.

FIG. 17 depicts a plan view of the objects of FIG. 16.

FIG. 18A is a side elevational sectional view of the objects of FIG. 16 at the indicated location of FIG. 17. FIG. 18B is an expanded axial sectional view of the objects of FIG. 16 at the indicated location of FIG. 17.

FIG. 19 is a perspective view of the assembled working element of the device of FIG. 10.

FIG. 20 is a plan view of the objects of FIG. 19.

FIG. 21 is a side elevational view of the objects of FIG. 19.

FIG. 22 is a side elevational sectional view of the objects of FIG. 19 at the indicated location of FIG. 20.

FIG. 23 is a perspective view of the objects of FIG. 19 with the active electrode partially deployed.

FIG. 24 is a plan view of the objects of FIG. 19.

FIG. 25 is a side elevational view of the objects of FIG. 19.

FIG. 26 is a side elevational sectional view of the objects of FIG. 19 at the indicated location of FIG. 24.

FIG. 27 is a perspective view of the objects of FIG. 19 with the active electrode fully deployed.

FIG. 28 is a plan view of the objects of FIG. 27.

FIG. 29 is a side elevational view of the objects of FIG. 27.

FIG. 30 is a side elevational sectional view of the objects of FIG. 27 at the indicated location of FIG. 28.

FIG. 31 is a side elevational sectional view of the distal portion of an alternate embodiment.

FIG. 32 is a side elevational sectional view of the distal portion of an alternate embodiment.

FIG. 33A is a side elevational sectional view of the distal portion of an alternate embodiment at the start of a cycle for incremental vaporization of a tissue mass. FIG. 33B is a side elevational sectional view of the distal portion of the objects of FIG. 33A with the active electrode deployed prior to incremental vaporization of a tissue mass. FIG. 33C is a side elevational sectional view of the distal portion of the objects of FIG. 33A at the completion of incremental vaporization of a tissue mass.

FIG. 34A is a side elevational sectional view of the distal portion of an introducer assembly used for insertion of an alternate embodiment for incremental vaporization of a tissue mass. FIG. 34B is a side elevational sectional view of the distal portion of an introducer sleeve and of an alternate embodiment for incremental vaporization of a tissue mass. FIG. 34C is a side elevational sectional view of the objects of FIG. 34B with the introducer sleeve retracted distally. FIG. 34D is a side elevational sectional view of the objects of FIG. 34B with the active electrode deployed at the start of incremental vaporization of a tissue mass. FIG. 34E is a side elevational sectional view of the objects of FIG. 34B at the completion of incremental vaporization of a tissue mass.

FIG. 35 is a schematic view of the distal portion of an alternate embodiment similar in construction to probe 1 as depicted in FIG. 2 but provided with both liquid and gaseous irrigation, as well as suction/aspiration.

### DETAILED DESCRIPTION OF THE PREFERRED EMBODIMENTS

The present invention constitutes a marked improvement in the field of electrosurgery, more particularly, to high efficiency surgical devices and methods which use radio frequency (RF) electrical power to ablate, denature, vaporize and remove all or part of a tissue mass, with or without externally supplied liquids or gases, as well as to thermally treat (i.e., cauterize, coagulate and form lesions) any neighboring, adjacent and/or remaining tissue.

Although any methods and materials similar or equivalent to those described herein can be used in the practice or testing of embodiments of the present invention, the preferred methods, devices, and materials are now described. However, before the present materials and methods are described, it is to be understood that this invention is not limited to the particular compositions, methodologies or protocols herein described, as these may vary in accordance with routine experimentation and optimization. It is also to be understood that the terminology used in the description is for the purpose of describing the particular versions or embodiments only, and is not intended to limit the scope of the present invention which will be limited only by the appended claims.

### Elements of the Present Invention

In the context of the present invention, the following definitions apply:

The words “a”, “an”, and “the” as used herein mean “at least one” unless otherwise specifically indicated.

In common terminology and as used herein, the term “electrode” may refer to one or more components of an electrosurgical device (such as an active electrode or a return electrode) or to the entire device, as in an “ablator electrode” or “cutting electrode”. Such electrosurgical devices are often interchangeably referred to herein as electrosurgical “probes” or “instruments”.

The present invention makes reference to an “active electrode” or “active element”. As used herein, the term “active electrode” refers to one or more conductive elements formed from any suitable metallic material, such as stainless steel, nickel, titanium, tungsten, and the like, connected, for example via cabling disposed within the elongated proximal portion of the instrument, to a power supply, for example, an externally disposed electrosurgical generator, and capable of generating an electric field.

The present invention makes reference to a “floating electrode” or “floating element”. As used herein, the term “floating electrode” refers to one or more conductive elements formed from any suitable metallic material, such as stainless steel, nickel, titanium, tungsten, and the like, that, while disconnected from any power supply, is nevertheless but capable of intensifying the electric field in proximity to the active electrode and aid in bubble retention when the instrument is used to vaporize tissue.

The present invention makes reference to a “return electrode”. As used herein, the term “return electrode” refers to one or more powered conductive elements formed from any suitable electrically conductive material for example metallic material, such as stainless steel, nickel, titanium, tungsten, aluminum and the like, to which current flows after passing from the active electrode(s) back to the electrical RF generator. One or more return electrodes may be positioned at a remote site on the patient's body (a configuration referred to herein as “monopolar”) or, alternatively, on the electrosurgical instrument itself (a configuration referred to herein as “bipolar”). In either context, for best results, it is preferable to position the return electrode in the vicinity of or proximate to the active electrode.

The present invention makes reference to “fluid(s)”. As used herein, the term “fluid(s)” refers to liquid(s), either electrically conductive or non-conductive, and to gaseous material, or a combination of liquid(s) and gas(es).

The term “proximal” refers to that end or portion which is situated closest to the user; in other words, the proximal end of an electrosurgical instrument of the instant invention will typically include the handle portion.

The term “distal” refers to that end or portion situated farthest away from the user; in other words, the distal end of an electrosurgical instrument of the instant invention will typically include the active electrode portion.

The present invention makes reference to the vaporization of tissue, more preferably a mass of soft tissue, even more preferably tumor tissue. As used herein, the term “tissue” refers to biological tissues, generally defined as a collection of interconnected cells that perform a similar function within an organism. Four basic types of tissue are found in the bodies of all animals, including the human body and lower multicellular organisms such as insects, including epithelium, connective tissue, muscle tissue, and nervous tissue. These tissues make up all the organs, structures and other body contents. The present invention is not limited in terms of the tissue to be treated but rather has broad application to the vaporization any target tissue with particular applicability to the ablation, destruction and removal of benign and cancerous tumors.

The instant invention has both human medical and veterinary applications. Accordingly, the terms “subject” and “patient” are used interchangeably herein to refer to the person or animal being treated or examined. Exemplary animals include house pets, farm animals, and zoo animals. In a preferred embodiment, the subject is a mammal.

Unless otherwise defined, all technical and scientific terms used herein have the same meaning as commonly understood by one of ordinary skill in the art to which this invention belongs. In case of conflict, the present specification, including definitions, will control.

### Utilities of the Present Invention

As noted above, the present invention is directed to high efficiency monopolar or bipolar electrosurgical instruments and methods which utilize radio frequency (RF) energy to destroy and remove tumors and soft tissues in situ, with or without externally supplied liquids, having particular utility in the context of oncology as well as general surgery.

Certain embodiments of the electrosurgical instrument of the present invention find particular utility in the treatment of tissue surfaces. Others are configured for sub-surface tissue treatment. Similarly, while some embodiments utilize the endogenous fluid of a “wet field” environment to transmit current to target sites, others require an exogenous irrigant. In certain embodiments, the “irrigant” (whether native or externally applied) is heated to the boiling point, whereby thermal tissue treatment arises through direct contact with either the boiling liquid itself or steam associated therewith.

To that end, the present inventors have discovered that under certain conditions and for certain tissue types, it is advantageous to minimize the amount of liquid present at the surgical site. Under these conditions it is beneficial not only to aspirate liquids and other ablation by-products from the site, but to also provide a gaseous “irrigant” to the site. The gaseous irrigant may be for example, dried air, nitrogen, argon, freon, helium, carbon dioxide (CO₂) or other suitable gas, either singly or in combination. In addition, liquids (either electrically conductive or non conductive) and gaseous irrigants, either singly or in combination may also be advantageously used as irrigant. Aspiration and irrigation through the ablating device may be balanced so as to maintain the ablation site at a slight positive pressure to optimize ablating conditions and/or maintain the volume of the cavity created by the vaporized tissue. Minimizing liquid at the ablation site simplifies the ablator design since current flows only from surfaces in contact with, or close proximity to, tissue, rather than from all uninsulated surfaces in contact with fluid at the site.

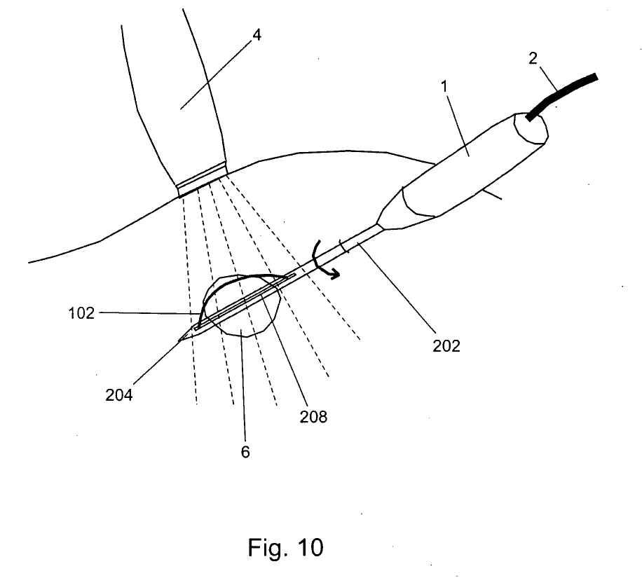

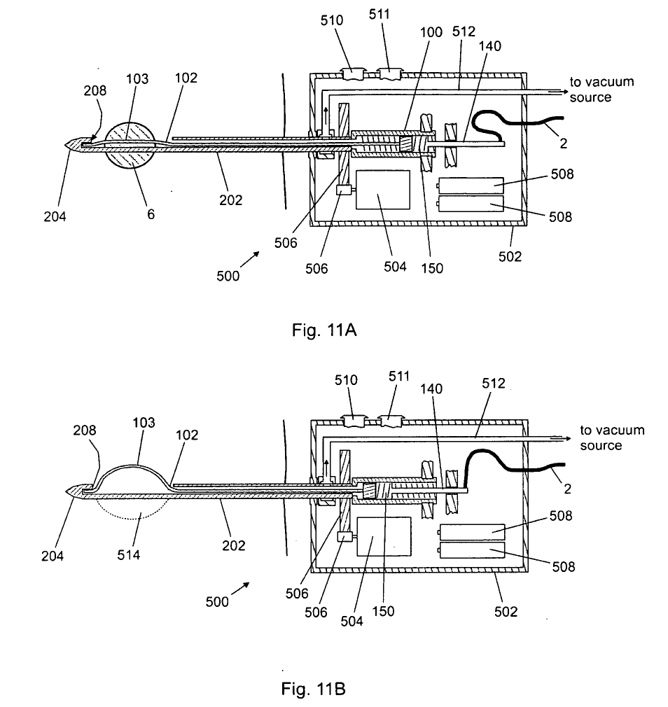

Liquids (either electrically conductive or non conductive) and gaseous irrigants, either singly or in combination may also be advantageously applied to devices for incremental vaporization of tissue as depicted in FIG. 10. Normal saline solution may be used. Alternatively, the use of low-conductivity irrigants such as water or gaseous irrigants or a combination of the two allows increased control of the ablating environment.

The electrosurgical devices of the present invention may be used in conjunction with existing diagnostic and imaging technologies, for example imaging systems including, but not limited to, MRI, CT, PET, x-ray, fluoroscopic, thermographic, photo-acoustic, ultrasonic and gamma camera systems. Such imaging technology may be used to monitor the introduction and operation of the instruments of the present invention. For example, existing imaging systems may be used to determine location of target tissue, to confirm accuracy of instrument positioning, to assess the degree of tissue vaporization (e.g., sufficiency of tissue removal), to determine if subsequent procedures are required (e.g., thermal treatment such as coagulation and/or cauterization of tissue adjacent to the target tissue and/or surgical site), and to assist in the atraumatic removal of the device.

As noted above, the electrosurgical devices of the present invention find utility in bulk or incremental tissue vaporization, more particularly in vaporization of tumor tissue, both benign and cancerous, with or without externally supplied fluids. Though the present invention is not particularly limited to the treatment of any one specific disease, body part or organ or the removal of any one specific type of tumor, the devices of the present invention nevertheless find particular utility in the treatment and removal of liver, breast, bladder, brain and spinal tumors, uterine fibroids, ovarian cysts, and colon polyps as well as the treatment of noncancerous conditions such as endometriosis.

### Illustrative Embodiments of the Present Invention

Hereinafter, the present invention is described in more detail by reference to the exemplary embodiments. However, the following examples only illustrate aspects of the invention and in no way are intended to limit the scope of the present invention. As such, embodiments similar or equivalent to those described herein can be used in the practice or testing of the present invention.

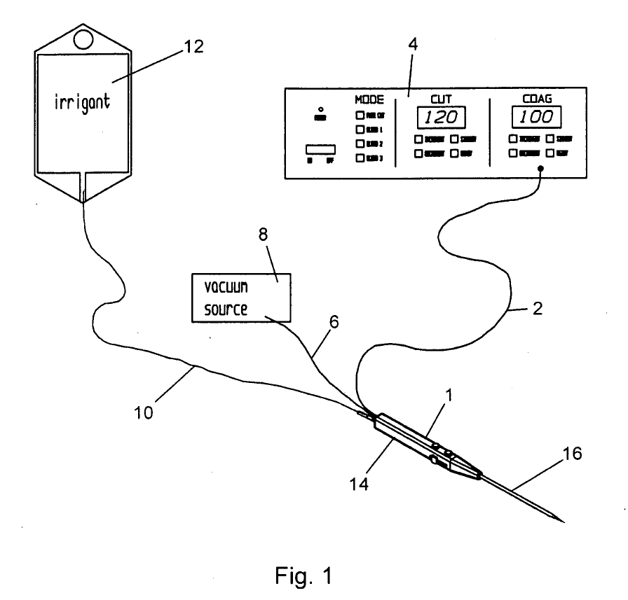

FIG. 1 depicts an electrosurgical system constructed in accordance with the principles of the present invention. Probe 1 is connected by cable 2 to electrosurgical generator 4. Probe 1 is connected by tubing 6 to vacuum source 8, and by tubing 10 to irrigant source 12. Probe 1 has a proximal portion 14 forming a handle, and an elongated distal portion 16 with distal end portion 17.

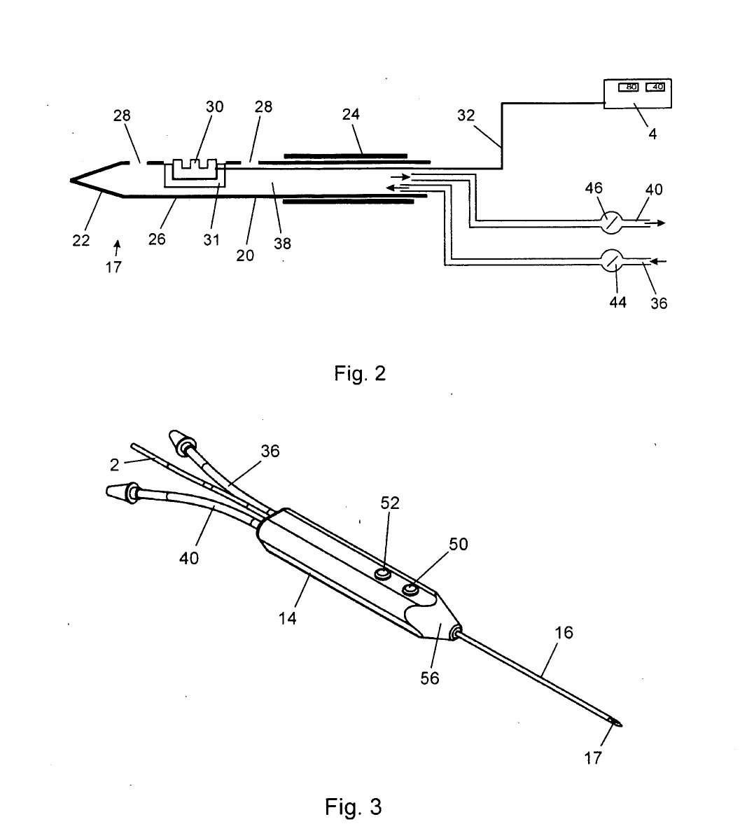

FIG. 2 is a schematic view of the distal portion of probe 1. Tubular member 20 with distal end portion 17 is made from a suitable electrically conductive material and has a pointed distal end 22 which facilitates penetration into tissue. The external surface of member 20 is covered by dielectric coating 24, with the exception of distal portion 26 which is uninsulated. Distal portion 26 has formed therein openings 28. Active electrode 30, surrounded by insulator 31 made from a suitable dielectric material, is connected by electrical cable 32 and means within proximal portion 14 and cable 2 to generator 4. Tube 36 conducts irrigant supplied by tubing 10 and means within proximal portion 14 from source 12 to lumen 38 of member 20. Tube 40, connected by means within proximal portion 14 and tubing 6 to vacuum source 8, provides a vacuum to lumen 38 of member 20. Valve 44 controls the flow of irrigant through tube 36. Valve 46 controls the supply of vacuum to lumen 38 by tube 40. Irrigant supplied to lumen 38 of member 20 flows through openings 28. Vacuum supplied to lumen 38 of member 20 causes irrigant and tissue vaporization byproducts to be aspirated from the site through openings 28.

FIG. 3 again depicts probe 1. Proximal portion 14 has near its distal end first button 50 labeled “ABLATE” and second button 52 labeled “COAG”, buttons 50 and 52 being connected by electrical cabling to generator 4 such that when button 50 is depressed RF power having a waveform suitable for vaporization of tissue is supplied to active electrode 30; depressing button 52 causes RF power suitable for coagulation of bleeding tissue to be supplied to active electrode 30. Tubes 36 and 40 pass from the proximal end 54 of proximal portion 14. Distal portion 16 protrudes from distal end 56 of proximal portion 14.

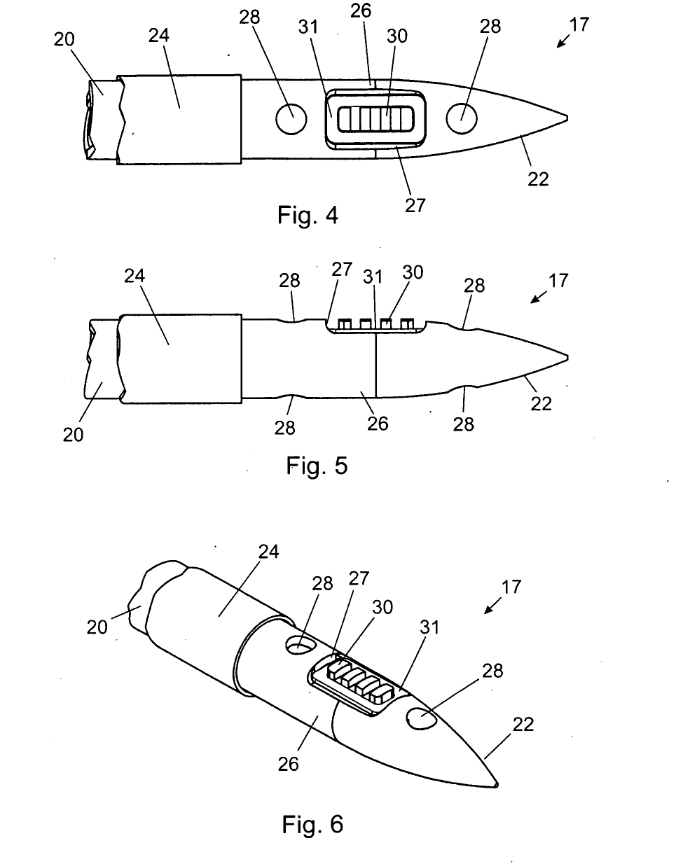

Referring now to FIGS. 4 through 6, which depict the distal end portion 17 of distal portion 16 of the probe of FIG. 1, dielectric coating 24 covers portion 16 except for uninsulated portion 26. Distal portion 17 has formed therein a short distance from pointed end 22 opening 28 in which are positioned insulator 31 and active electrode 30. Electrode 30 has a plurality of protuberances formed on its distal surface. During use, uninsulated portion 26 of member 20 acts as a floating electrode, a portion of the current flowing from active electrode 30 to tissue, through tissue to regions of portion 26 adjacent to active electrode 30 which are in high-potential regions of the electric field, through portion 26 to lower potential regions of the electric field, and from portion 26 to adjacent irrigant and tissue.

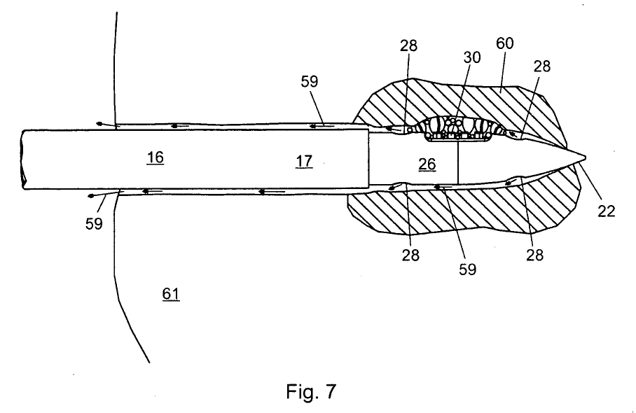

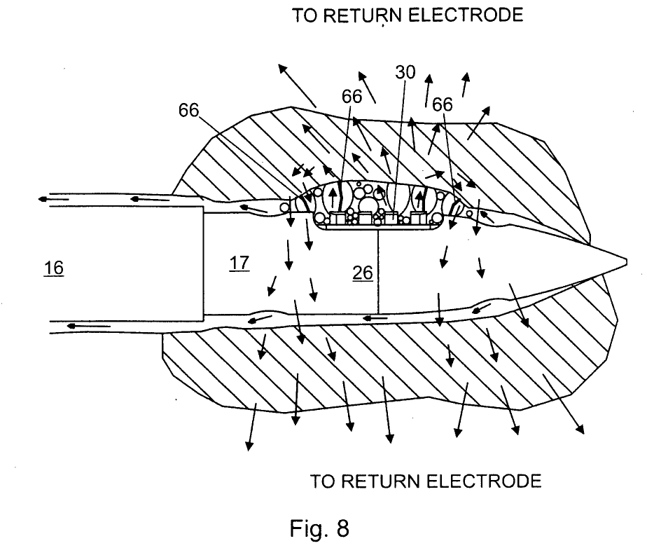

Referring to FIGS. 7 and 8, which depict the probe 1 of FIG. 1 treating a tumor 60 surrounded by tissue 61, distal end 22 penetrates tissue 61 under image guidance until uninsulated portion 26 is within the tumor. Valve 44 (FIG. 2) is opened allowing irrigant 59 under pressure to flow to the site via openings 28, irrigant 59 flowing proximally between distal portion 16 (FIG. 3) and tumor 60 and tissue 61 after exiting the openings. The irrigant 59 may be electrically conductive or nonconductive. Mixing of irrigant 59 with bodily fluids and contaminants at the site will result in a local environment filled with liquid having some degree of electrical conductivity, thereby allowing current to flow through the liquid. During use, current flows from active electrode 30 to a return electrode and therefrom to generator 4. A portion of the current flows from active electrode 30, through the surrounding liquid to tumor 60, and finally through tumor 60 and tissue 61 to the return electrode. Another portion of the current flows from active electrode 30 through the surrounding liquid to tumor 60, through tumor 60 to regions of tumor 60 that are in close proximity to portions of uninsulated portion 26 in close proximity to active electrode 30, through the surrounding liquid to portion 26. This current then flows through portion 26 to regions of portion 26 which are in lower potential portions of the electric field, and therefrom through tumor 60, tissue 61 and liquid in contact with portion 26 to the return electrode. The instrument is energized, causing boiling of irrigant 59 and formation of bubbles at active electrode 30 and at the edges of regions of uninsulated portion 26 in close proximity to active electrode 30. Arcs 66 form within some of the bubbles, the arcing being primarily between active electrode 30 and adjacent tumor tissue, and between regions of uninsulated portion 26 in close proximity to active electrode 30 and adjacent tumor tissue. A volume of tumor tissue is vaporized. After a brief period of activation, valve 46 is opened so as to aspirate debris and heated fluid from the site. When the site has been cleared, distal end 17 may be repositioned and the preceding steps may be repeated to vaporize another volume of tissue. The process may be repeated until the entire tumor has been vaporized. Optionally, after vaporization of the desired tissue is completed, probe 1 can be energized at a lower power level, one that is insufficient for tissue vaporization but nevertheless heats tissue in proximity to distal end 22 so as to thermally necrose (or coagulate or cauterize) any remaining tumor tissue.

In an alternate embodiment, a switching means is provided in proximal handle portion 14 of probe 1, or within generator 4 to allow RF energy to be connected to either active electrode 30 or uninsulated portion 26. During penetration of distal portion 16 into tissue 61 and tumor 60, RF energy is supplied to uninsulated portion 26 so that tissue is vaporized by distal end 22. When uninsulated portion 26 is positioned within tumor 60, portion 26 is disconnected from generator 4 and active electrode 30 is connected to generator 4. Thereafter the function of probe 1 is the same as in the previous embodiment.

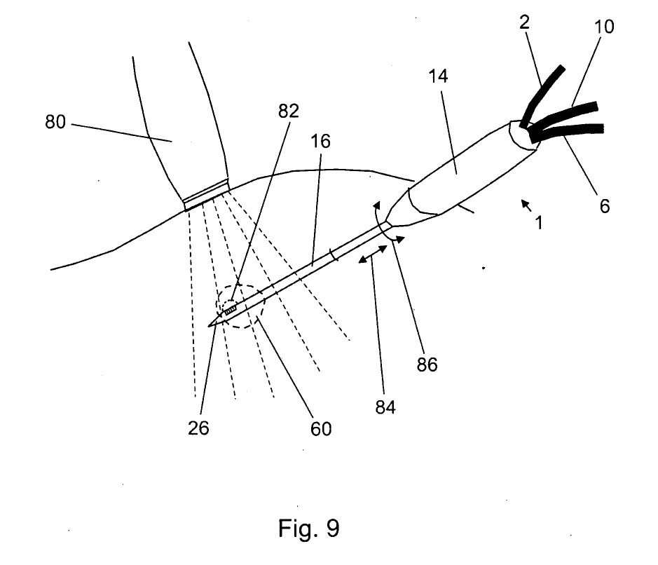

FIG. 9 depicts probe 1 in use positioned by imaging system 80 so that distal end 26 is positioned slightly distal to tumor 60. Volume 82 has been vaporized. Probe 1 may be repositioned by rotation 86 about its axis, and by axial translation 84 to vaporize other portions of tumor 60. The process is repeated until all of tumor 60 has been vaporized.

Numerous adaptations and modifications may be made to the embodiments herein disclosed without departing from the principles of the incident invention, which is a device and method for vaporizing a tumor or tissue using RF energy. For example, the location, shape, size, geometry and material for the floating, active and return electrodes, as well as the insulators may deviate substantially from the preferred embodiment described here. In preferred embodiments, the electric field intensity is increased in proximity to active electrode 30 by a floating electrode. In another preferred embodiment, the floating electrode is integral with distal portion 16 of probe 1. In other embodiments it is not integral. In another preferred embodiment, active electrode 30 is laterally positioned; in other embodiments the active electrode faces distally, or is angled between lateral and distal positions. Probe 1 may be used with a remotely located return electrode (i.e., in a monopolar configuration). Other embodiments contemplate the positioning of a return electrode on the probe itself (i.e., in a bipolar configuration), preferably in the vicinity of or proximate to the distal portion of the active electrode.

FIG. 10 depicts an electrosurgical device formed in accordance with the principles of the present invention. Device 1 is connected to a suitable electrosurgical generator (not shown) by cord 2. Using imaging system 4, mass 6 is located and distal portion 202 is inserted and positioned such that distal end 204 is distal to the distal margin of the mass. The device is activated causing distal portion 202 to rotate while electrode 102 is deployed from slot 208, each rotation causing an increase in the extension of electrode 102, RF energy being supplied to electrode 102. Tissue contacted by electrode 102 is instantly vaporized. When electrode 102 is fully deployed, mass 6 is completely vaporized. Electrode 102 is then retracted into distal portion 202 and the device removed from the patient.

FIGS. 11A and 11B diagrammatically depict a device for incremental vaporization of a tumor or other tissue mass. Device 500 has a handle portion 502 and a rotatable portion 200 having an elongated distal portion 202. Distal portion 200 has positioned within it inner assembly 100 having an elongated electrode portion 102. Elongated electrode piece 102 together with distal portion 202 of outer assembly 200 may be rotated about the axis of portion 202. Handle portion 502 has a motor 504 coupled by gears 506 to outer assembly 200 so as to cause rotation of distal portion 202 and elongated electrode 102. Batteries 508 are connected to motor 504 so as to cause rotation in a first direction when first button 510 is depressed. Depressing button 510 also causes simultaneous activation of the electrosurgical generator so that RF energy is supplied to electrode 102 by cable 2. Rotation of assembly 200 causes threaded portion 150 of pusher 140 to be threaded into outer assembly 200 so as to displace the proximal portion of electrode 102 distally, the displacement increasing with each rotation of assembly 200. Displacement of the proximal portion causes the distal portion 103 of 102 to deform so as to protrude from slot 208 in the distal end of portion 202, the protrusion increasing with each rotation of assembly 200. FIG. 11A depicts device 500 prior to activation of the device, as inserted into a patient so as to remove tumor 60. When device 500 is activated, portion 202 rotates with the radial displacement of the proximal portion of electrode 102 increasing with each rotation. At a location within tumor 60, tissue is incrementally vaporized with each rotation until removal of the tumor is complete. During activation, vaporization byproducts are aspirated from the site via path 512 in handle 502. FIG. 11B depicts device 500 at the completion of removal of tumor 60 having formed void 514 within the tissue. Depressing second button 511 causes reversal of the motor 504 so as to retract the proximal end of electrode 102 causing distal portion of electrode 102 to be withdrawn into slot 208 of elongated member 202 so that the device can be withdrawn from the site. In an alternate embodiment, second button 511 causes disengagement of the threaded portions of inner assembly 100 and outer assembly 200 so that electrode 102 can be withdrawn into slot 208 to allow withdrawal of the device from the site.

Device 500 employs a combination rotary and translational motion to incrementally vaporize a volume of tissue. The rotary and translational motion is produced mechanically by threaded elements within handle 502, their function being an important part of this invention. Accordingly, subsequent figures and descriptions will focus on these elements. It will be understood that device 500 uses a motor, gears, a power source and activation means well understood in the art, and there eliminated from subsequent figures for clarity.

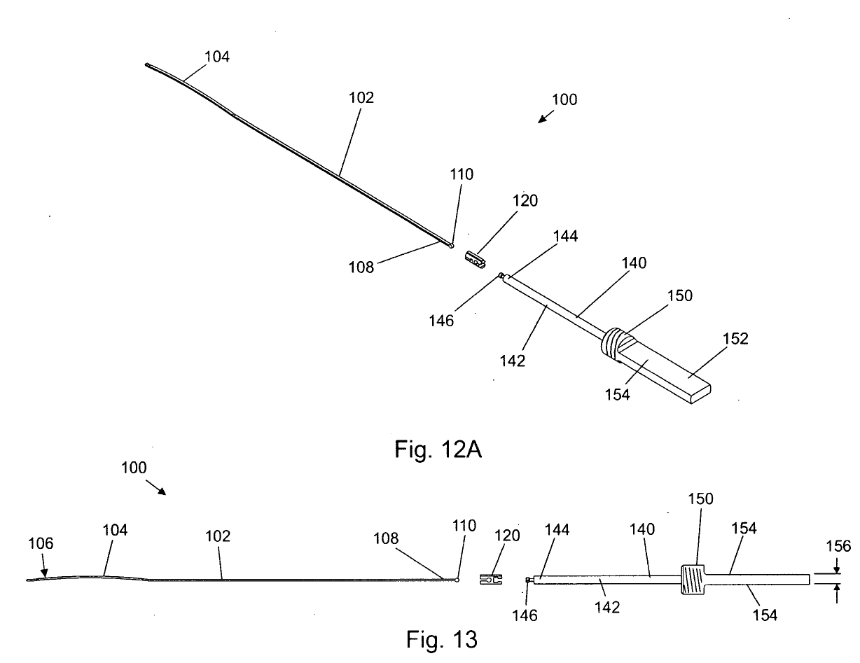

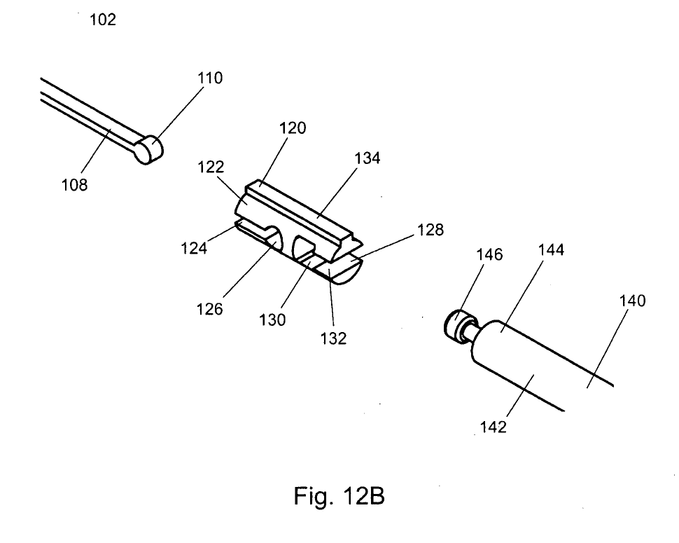

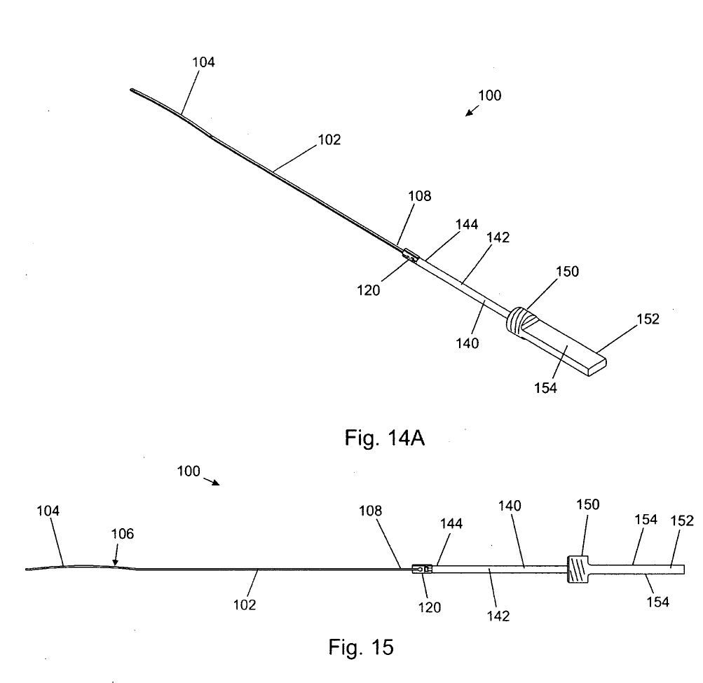

Referring to FIGS. 12A, through 15 depicting the inner assembly of a device 500 for incremental vaporization of tissue masses, and formed in accordance with the principles of this invention, inner assembly 100 has an elongated electrode piece 102, a link piece 120, and a proximal driver piece 140. Electrode piece 102 has a distal portion 104 formed to a predetermined radius 106, and a proximal end 108 having formed thereon cylindrical portion 110. Link piece 120 has a distal portion 122 in which is formed slot 124 having formed therein a cylindrical portion 126 formed to receive cylindrical portion 110 of proximal end 108 of electrode 102. Link piece 120 has a proximal end in which is formed slot 128 having formed therein distal portion 130 greater in width than proximal portion 132 of slot 128. Link 120 has formed thereon axial key 134. Driver 140 has an elongated cylindrical distal portion 142 having at its distal end 144 protruding portion 146. Protruding portion 146 has a proximal cylindrical portion and a distal cylindrical portion, the distal and proximal portions of protruding portion 126 being configured such that portion 126 may be positioned within proximal slot 128 of link piece 120. Driver 140 has a threaded medial portion 150, and an elongated proximal portion 152 having formed thereon parallel planar surfaces 154 displaced from each other distance 156.

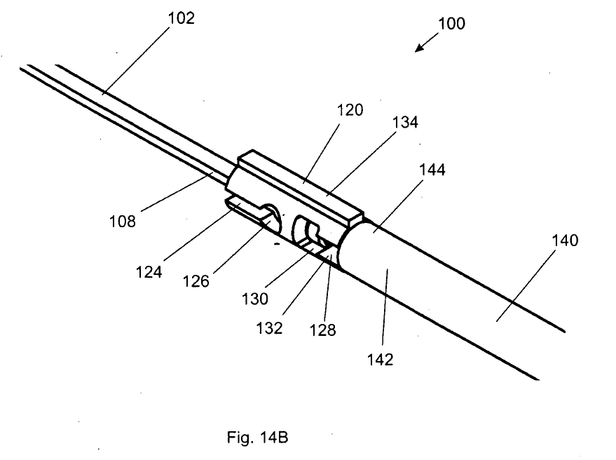

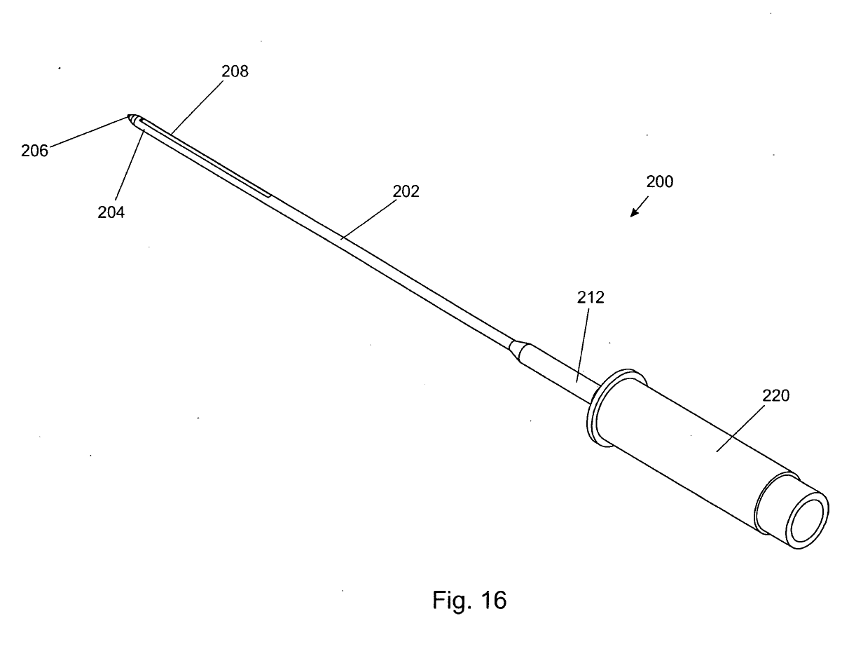

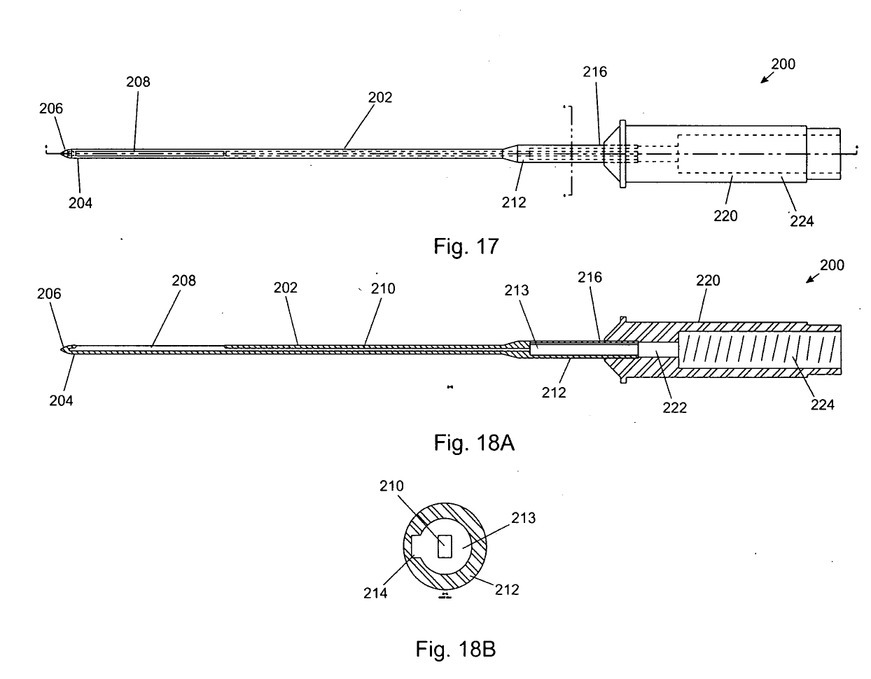

FIGS. 16 through 18B depict the outer assembly of a device for incremental vaporization of tissue masses, and formed in accordance with the principles of this invention. Outer assembly 200 has an elongated distal portion 202 having a distal end 204 with a sharpened portion 206 and laterally facing slot 208 formed adjacent thereto. Rectangular lumen 210 at its distal end intersects slot 208, and its proximal end intersects tubular portion 212 having a lumen 213 of a diameter slightly larger than the outer diameter of link piece 120, and a keyway 214 slightly greater in width than key 134 of link piece 120. Proximal housing 220 is mounted to proximal end 216 of elongated distal portion 202 which is pressed into distal lumen 222 of proximal housing 220. Housing 220 is tubular in structure having a distal end lumen 222, and a proximal internally threaded portion 224, the threads of which match those of threaded medial portion 150 of driver 140. Distal portion 202 is preferably formed from a dielectric material. In other embodiments, distal portion 202 may be formed from a non-dielectric material, though covered with a dielectric coating.

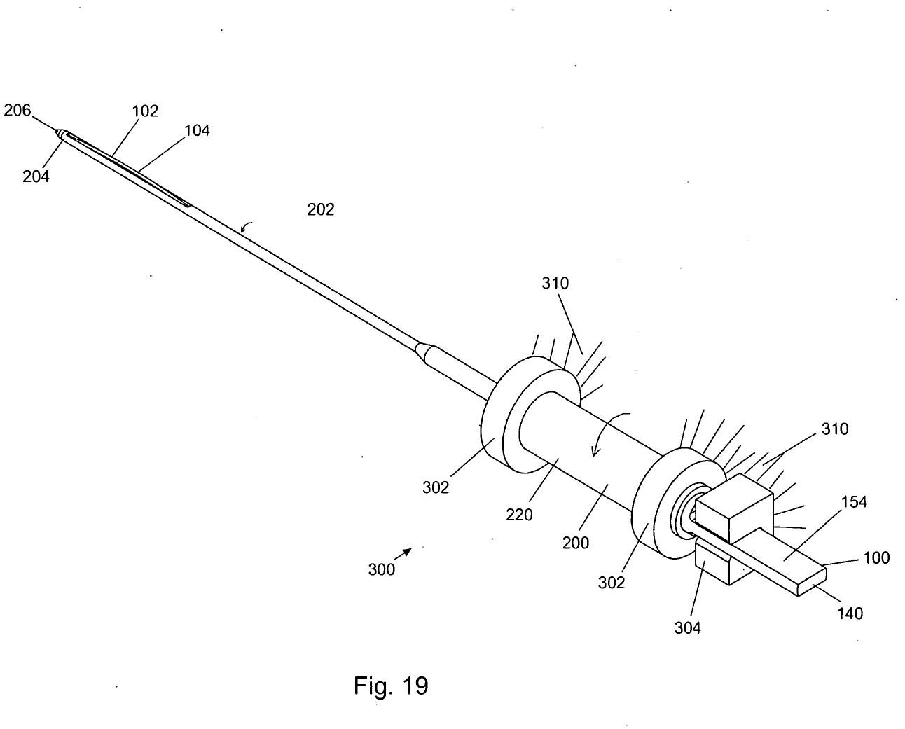

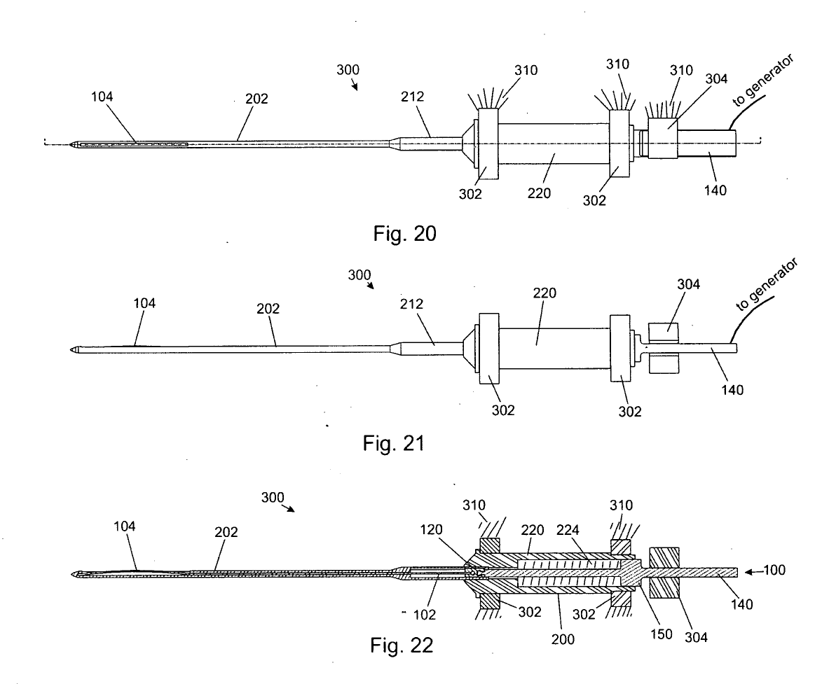

FIGS. 19 through 22 depict inner assembly 100 and outer assembly 200 assembled together to form working assembly 300 for an electrosurgical device formed in accordance with the principles of this invention. The complete device is not depicted. Rather, the working elements of the electrode which incrementally vaporizes a tissue mass are depicted. These elements are part of a device assembly which also comprises a motor which imparts rotational motion to outer assembly 200, a conductive means which supplies RF energy to inner assembly 100, an optional aspiration means for removing vaporization products from the site, and a housing which is held in the hand of the user. Housing 220 is rotatably mounted in bearings 302 which are, in turn, mounted in housing portions 310. Elongated proximal portion 152 of driver 140 axially slidably engages slotted portion 304 of housing portion 310. When the device is activated, rotary motion is imparted to proximal housing 220 of outer assembly 200 by an electric motor, not shown, resulting in rotation of outer assembly 200, elongated electrode 102 and link 120 because of engagement of key 134 of link 120 and keyway 214 of proximal portion 212 of distal portion 202. Driver 140 is prevented from rotating by slotted portion 304 of housing 310. Relative rotational motion between proximal housing 224 of outer assembly 200 and threaded medial portion 150 of driver 140 causes driver 140 to advance distally into proximal housing threaded portion 224. Advancing driver 140 distally causes elongated distal portion 142 of driver 140 to displace link 120 and proximal end 108 (FIG. 12B) of electrode 102 distally. Advancing the proximal end of electrode 102 distally causes distal portion 104 of electrode 102 to deform so as to protrude through slot 208 in distal portion 202 of outer assembly 200.

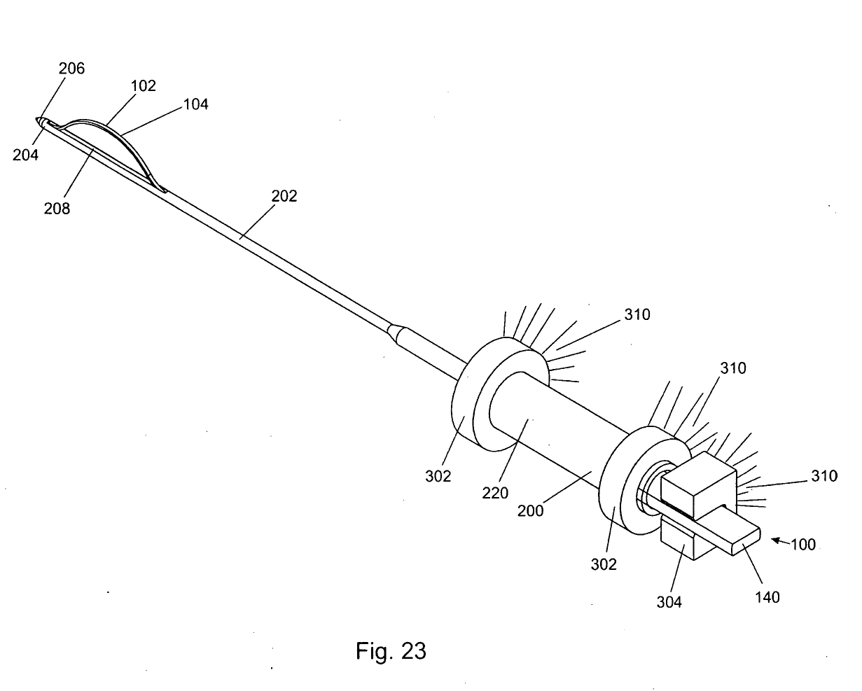

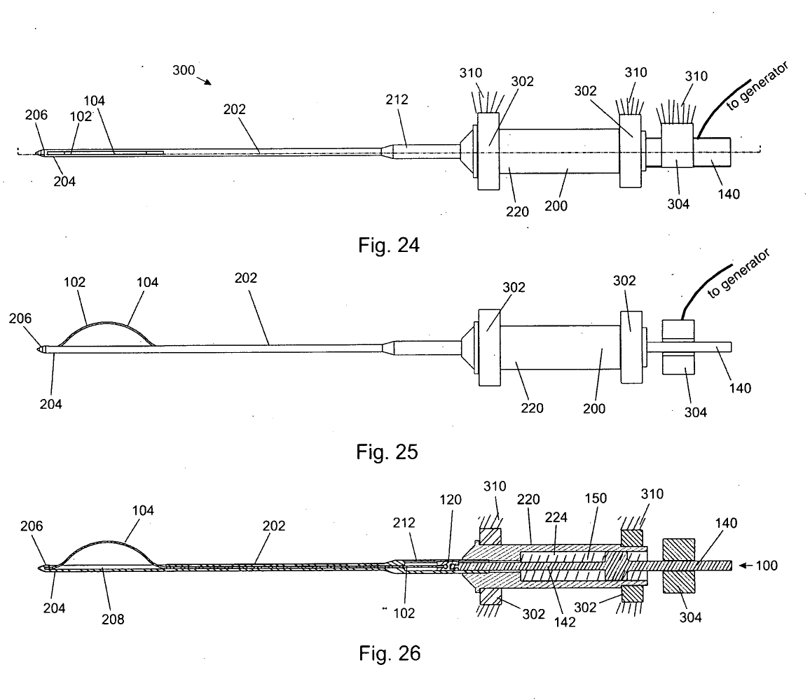

FIGS. 23 through 26 depict assembly 300 with driver 140 at the approximate mid-point of its travel in threaded portion 224 of proximal housing 220 of outer assembly 200. Axial displacement of proximal end 108 (FIG. 12B) of electrode 102 relative to outer tubular member 202 results in deformation of distal portion 104 of electrode 102, the deformation being proportional to the displacement of driver 140, and the rate of displacement being determined by the rotational speed of outer assembly 200 and the pitch of the thread of threaded portion 224 of housing 220.

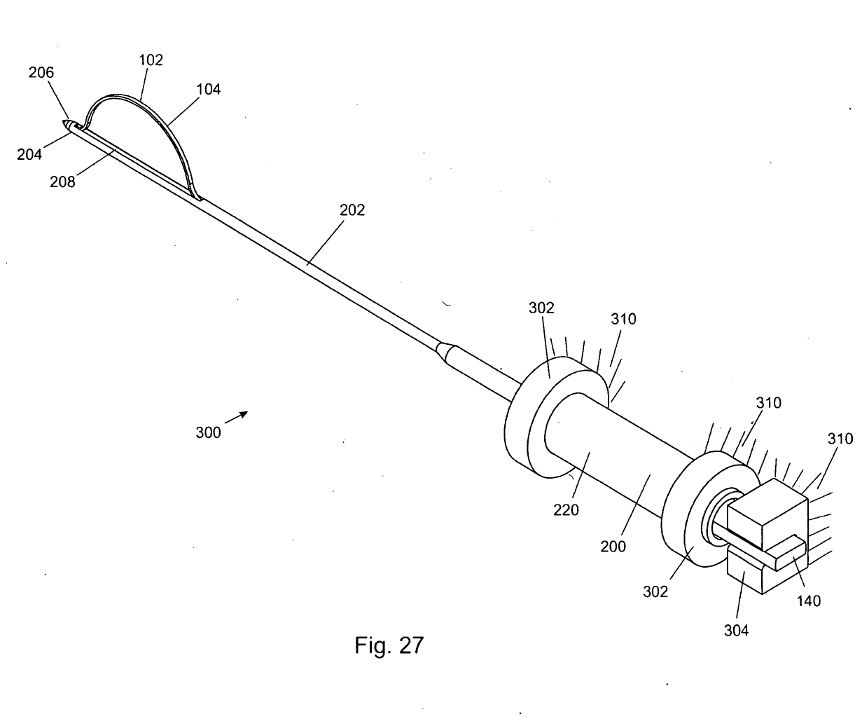

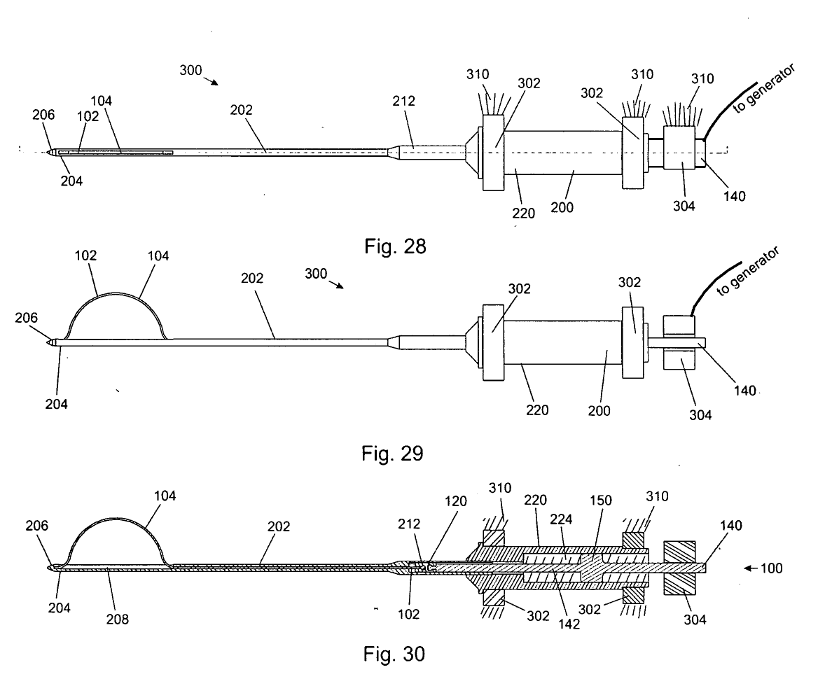

FIGS. 27 through 30 depict assembly 300 with driver 140 at the distal limit of its travel in threaded portion 224 of proximal housing 220 of outer assembly 200. Electrode 102 has been fully deployed from slot 208. Vaporization of tissue by electrode 102 which has undergone progressive deformation during rotation of outer assembly 220 results in the creation of a approximately spherical void within the tissue, the radius of the void being determined by the maximum deflection of distal portion 104 of electrode 102. When vaporization of the volume is completed, rotation of outer assembly 200 is reversed causing distal portion 104 of electrode 102 to be withdrawn back into slot 208 so that the device distal portion can be removed from the patient.

Tissue at a location treated using device 500 is incrementally vaporized. That is, each rotation of the distal portion of the device causes electrode 102 to vaporize an additional layer of tissue at a location.

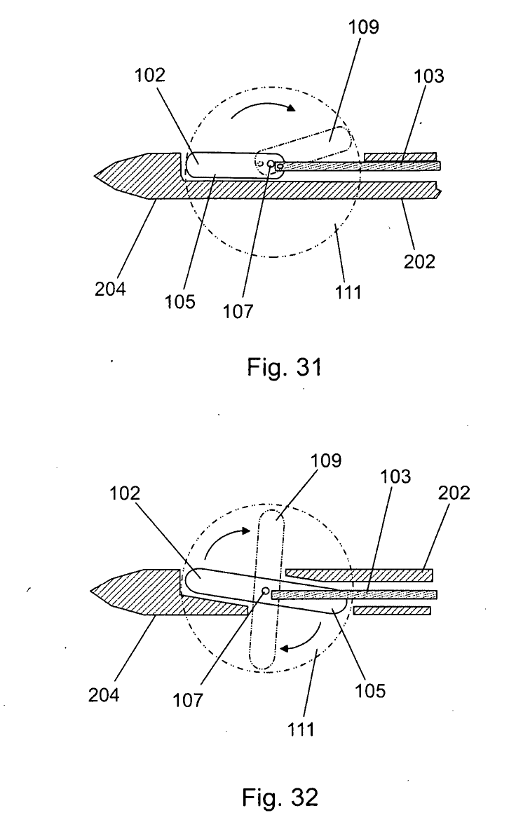

Other configurations of electrode 102 are anticipated in which the electrode is not elastically deformed, but in which a distal movable element functions as the electrode. FIG. 31 depicts one such configuration. Electrode 102 is pivotally mounted, it's angular position being controlled by control link 103. With electrode 102 in a first position 105, distal end 204 is positioned as shown in FIG. 10. Device 500 is activated causing distal portion 202 to rotate. With each rotation control link 103 is advanced distally causing electrode 102 to rotate about pivot 107 until second position 109 is reached creating void 111. A second embodiment employing a pivot is depicted in FIG. 32. In this embodiment electrode 102 has two active portions such that void 111 can be created by rotating electrode 102 about pivot 107 from first position 105 to second position 109 with less angular rotation of element 102 about pivot 107.

In other embodiments the mechanism of handle 502 is modified so that the axial position of driver 140 is fixed and distal portion 202 moves axially from a first position to a second position during use. In these embodiments electrode 102 is deployed following insertion of the device so as to be displaced radially from distal portion 202. After this, device 500 is activated causing rotation of portion 202 and deployed electrode 102, and axial translation of these elements. The void created by the combined rotational and axial motions of the electrode create a void having a more or less cylindrical shape, the radius of the cylinder being determined by the radial displacement of electrode, and the length of the cylinder being determined by the axial travel of the electrode when energized.

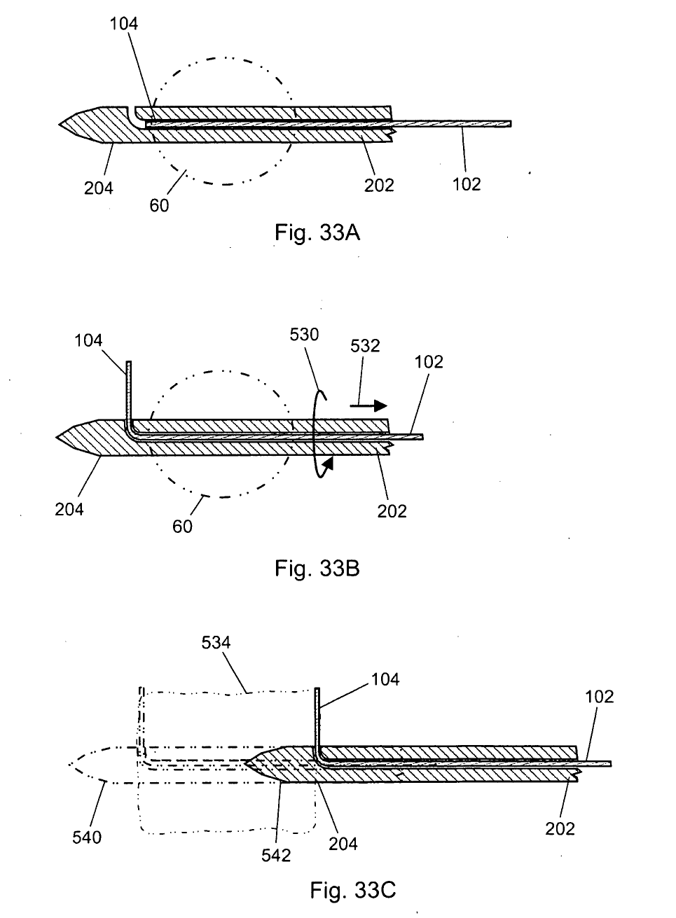

FIGS. 33A-33C depict the distal portion of a device for incremental vaporization of a tissue mass using combined rotational and axial motion of an active electrode. Referring to FIG. 33A, distal end 204 of elongated portion 202 is positioned distal to mass 60 which is to be removed by incremental vaporization. Electrode 102 is retracted proximally to a first position. In FIG. 33B, electrode 102 has been advanced distally to a second position such that its distal end 104 protrudes radially outward from portion 202. Device 500 is then activated causing simultaneously rotational motion 530 and axial motion 532 while RF energy is supplied to electrode 102. Distal portion 202 is moved proximally from its initial first position 540 to a second position 542. FIG. 33C depicts the site after completion of incremental vaporization of mass 60. Void 534, created by device 500 has a more or less cylindrical shape. At the completion of the cycle, electrode 102 is retracted into member 202 and the assembly withdrawn from the site.

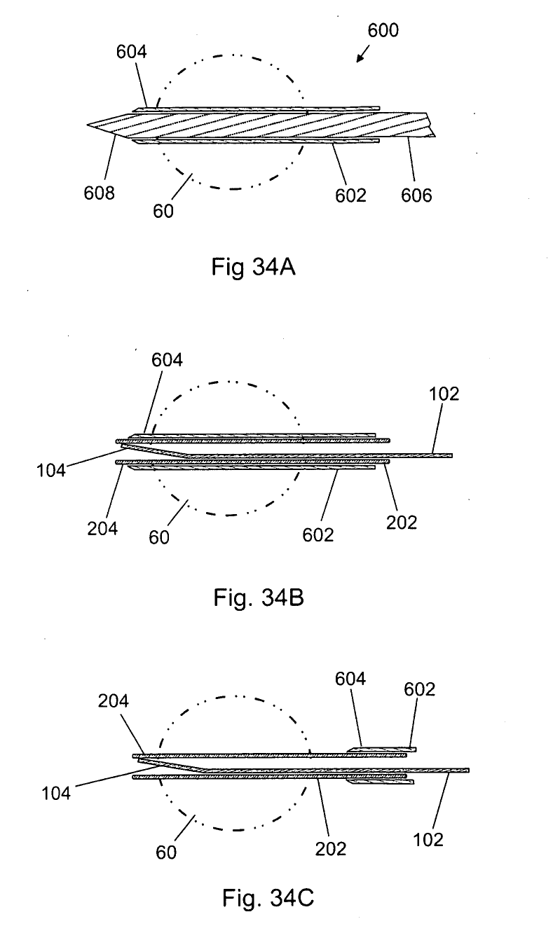

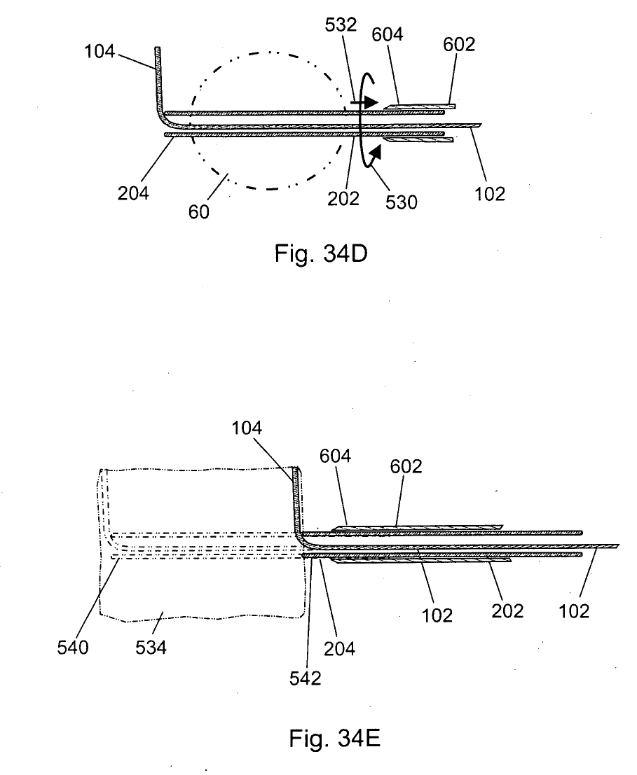

In other embodiments, which use combined rotational and axial motion to incrementally vaporize a tissue mass, distal end 204 of elongated member 202 is introduced to the site of the mass to be removed via a cannulated introduction device. Such devices are well known in the art and allow instruments having blunt distal ends to be introduced into structures within the body. Because sharpening of the distal end is not necessary, active electrode 102 can be deployed from the distal end of the device allowing additional electrode configurations to be utilized. FIGS. 34A through 34E depict an alternate embodiment for use with an introduction device.

Referring to FIG. 34A, introducer 600 has sleeve 602 with a distal end 604, and an inner member 606 having a sharpened distal end 608. Introducer 600 is inserted into the patient and positioned such that distal end 604 is distal to tissue mass 60 which is to be removed by incremental vaporization. In 34B inner member 606 has been removed and replaced by distal portion 202 of incremental vaporization device 500. In 34C, sleeve 602 has been retracted. FIG. 34D depicts the distal portion of device 500 with distal portion 104 of active electrode 102 deployed form distal end 204 of outer member 202. Electrode 102 is formed so that distal end 104 elastically bends as depicted upon deployment from distal end 204 of 202. Device 500 is then activated causing simultaneously rotational motion 530 and axial motion 532 while RF energy is supplied to electrode 102. Distal portion 202 is moved proximally from its initial first position 540 to a second position 542. FIGS. 33C and 34E depicts the site after completion of incremental vaporization of mass 60. Void 534, created by device 500 has a more or less cylindrical shape. At the completion of the cycle, electrode 102 is retracted into member 202 and the assembly withdrawn from the site.

In the embodiment of FIGS. 34A through 34E, active electrode 102 is deployed by elastic bending of distal portion 104. In other embodiments the bend is inelastic, distal portion 104 being formed by forming means in the distal end 204 of outer member 202. The forming means may comprise a curved forming channel which inelastically forms portion 104 to a predetermined shape as it passes through the forming channel of member 202. In other embodiments portion electrode 102 is a pivoting member similar to that shown in FIG. 31, electrode 102 being deployed from the distal end of outer member 202.

While the cycle time for incrementally vaporizing a tissue mass is much shorter than that of previous methods, it may prove difficult for the physician to maintain the position of the distal portion 202 of the device during treatment. Maintaining this position is particularly important for embodiments which use axial motion of the distal portion to incrementally vaporize the mass. The inventors anticipate embodiments in which an external means is utilized to maintain the device position during treatment. The means may comprise a positionable rigid structure which holds the device during treatment.

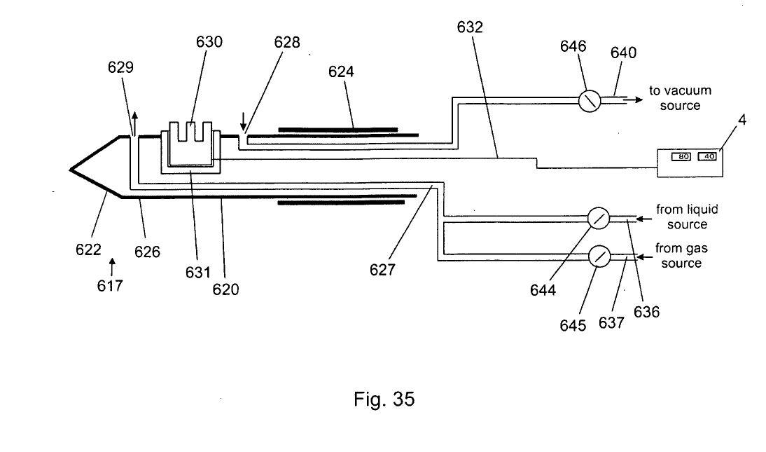

FIG. 35 is a schematic view of the distal portion of an alternate embodiment similar in construction to probe 1 as depicted in FIG. 2 but provided with both liquid and gaseous irrigation. Tubular member 620 with distal end portion 617 is made from a suitable electrically conductive material, has a pointed distal end 622 which allows penetration of tissue. The external surface of member 620 is covered by dielectric coating 624 except for distal portion 626 which is uninsulated. Distal portion 626 has formed therein first openings 628 and second openings 629. Active electrode 630, surrounded by insulator 631 made from a suitable dielectric material, is connected by electrical cable 632 and means within proximal portion 14 and cable 2 to generator 4 (FIG. 1). Tube 640, connected by means within proximal portion 14 and tubing 6 to vacuum source 8, provides a vacuum to first openings 628 of member 620. Valve 646 controls the supply of vacuum to lumen first openings 628 by tube 640. Vacuum supplied to first openings 628 of member 620 causes irrigant and tissue vaporization byproducts to be aspirated from the site through openings 628. Tube 627, in communication with second openings 629, supplies irrigant to the site, the irrigant being either liquid controlled by valve 644 and supplied via tube 636 from an external source, or a gaseous irrigant controlled by valve 645 and supplied via tube 637 from an external source, or a combination of liquid and gaseous irrigant. Valves 644 and 645 control the flow rates of the irrigants, and control the relative proportions of liquid and gaseous irrigants when used in combination. By controlling the relative flow rates of the irrigant(s) and the aspiration flow by valve 646, it is possible to increase the pressure at the site slightly above atmospheric so as to aid in maintaining the void formed by the vaporization of tissue, and to better control the ablating conditions at the site.

Liquid and gaseous irrigants, either singly or in combination may also be advantageously applied to devices for incremental vaporization of tissue as depicted in FIG. 10. Normal saline solution may be used. Alternatively, the use of low-conductivity irrigants such as water or gaseous irrigants or a combination of the two allows increased control of the ablating environment. In particular, decreasing the presence of conductive liquid at the site simplifies insulation of the device since current flows only from portions of the active electrode surfaces which are in contact with, or close proximity to tissue.

### INDUSTRIAL APPLICABILITY

The minimally invasive monopolar and bipolar electrosurgical instruments of the present invention find utility in the area of remote vaporization of tumor tissues, with or without externally supplied conductive or non-conductive liquids (i.e., in the context of both wet and dry field electrosurgery), with or without additional and/or automated support means (e.g., external stabilizing hardware, robotic control means, etc.).

All patents and publications mentioned herein are incorporated by reference in their entirety. Nothing herein is to be construed as an admission that the invention is not entitled to antedate such disclosure by virtue of prior invention.

While the invention has been described in detail and with reference to specific embodiments thereof, it is to be understood that the foregoing description is exemplary and explanatory in nature and is intended to illustrate the invention and its preferred embodiments. Through routine experimentation, one skilled in the art will readily recognize that various changes and modifications can be made therein without departing from the spirit and scope of the invention.

Other advantages and features will become apparent from the claims filed hereafter, with the scope of such claims to be determined by their reasonable equivalents, as would be understood by those skilled in the art. Thus, the invention is intended to be defined not by the above description, but by the following claims and their equivalents.

What is claimed:

1. An electrosurgical device for incremental vaporization of a mass of tissue at a target site in the body of a patient, said device comprising:
a. a proximal portion comprising a proximal control housing handle configured for connection to a power source;
b. a distal portion comprised of an outer assembly and an inner assembly;
c. wherein said outer assembly includes an elongate shaft having a an elongate lumen terminating in a distal opening and a distal tip that is sufficiently sharp to penetrate said target tissue and permit the positioning of said distal opening at said target site;
d. wherein said inner assembly comprises a proximal driver piece linked to at least one elongate active electrode connected to a power source;
e. wherein rotation of said proximal control housing about the longitudinal axis of said elongate shaft results in the passage of at least a distal portion of said active electrode through said distal opening and the radial projection of said distal portion into said mass of tissue.

2. The electrosurgical device of claim 1, wherein the dimension of said radial projection increases with each rotation of said assembly.

3. The electrosurgical device of claim 1, further wherein said outer assembly and said inner assembly are coordinated in such a fashion that relative rotational motion between said proximal control housing and said proximal driver piece causes the driver piece to advance distally which, in turn, causes the proximal end of said active electrode to advance distally which, in turn, causes a distal portion of said active electrode to pass through said distal opening.

4. The electrosurgical device of claim 1, wherein said distal opening comprises a laterally facing slot.

5. The electrosurgical device of claim 1, wherein said shaft is provided with multiple openings at the distal end.

6. The electrosurgical device of claim 1, wherein said active electrode comprises an elastically deformable wire element having a pre-formed distal loop segment, wherein rotation of said proximal control housing about the longitudinal axis of said shaft results in the deformation of the distal end of said active electrode, causing said pre-formed loop segment thereof to radially protrude through said distal slot, the dimension of said radial protrusion increasing with each rotation of said assembly.

7. The electrosurgical device of claim 1, wherein said active electrode comprises at least one pivotally mounted distal portion, the angular position of which is controlled by the proximal driver piece, wherein rotation of said proximal control housing about the longitudinal axis of said shaft results in the axial displacement of the proximal driver piece in the distal direction, which, in turn, causes the at least one distal portion to rotate and project out through said distal slot.

8. The electrosurgical device of claim 7, wherein said active electrode comprises a series of pivotally mounted distal portions, wherein a first rotation of said proximal control housing about the longitudinal axis of said shaft causes a first distal-most pivotally mounted distal portion to rotate and project out through said distal slot and a second rotation of said proximal control housing about the longitudinal axis of said shaft causes a second pivotally mounted distal portion to rotate and project out through said distal slot.

9. The electrosurgical device of claim 1, further comprising means for automating the incremental rotary motion imparted to the proximal control housing.

10. The electrosurgical device of claim 1, wherein said automating means comprises an electric motor.

11. The electrosurgical device of claim 1, wherein the proximal control housing is coupled to the proximal drive piece of said inner assembly by means of mating screw threads.

12. The electrosurgical device of claim 11, wherein the radial projection of distal portion of the active electrode is proportional to the axial displacement of the proximal driver piece, further wherein the rate of displacement is determined by the rotational speed of outer assembly and the pitch of the mating screw threads.

13. The electrosurgical device of claim 1, wherein said tissue comprises tumor tissue.

14. The electrosurgical device of claim 1, further comprising a means for supplying an irrigant to the target site.

15. The electrosurgical device of claim 14, wherein said irrigant is selected from a liquid, a gas or a combination thereof.

16. The electrosurgical device of claim 1, further comprising a means for aspirating vaporization by-products from the target site.

17. The electrosurgical device of claim 1, further comprising a means for switching from a first power level, sufficient to provide arcing and vaporization of tissue at a target site and a second power level wherein arcing and vaporization of tissue do not occur but yet is sufficient to thermally treat tissue adjacent to the target site.

18. The electrosurgical device of claim 1, wherein said outer assembly is comprised of two separate components: (a) an elongate shaft comprising an introducer sleeve and (b) a sharp-tipped inner member slidably received within the elongate lumen of said shaft and extending out said shaft distal opening.

19. The electrosurgical device of claim 1, further comprising a return electrode.

20. The electrosurgical device of claim 19, wherein said return electrode is proximate to the distal portion of said at least one active electrode.

21. The electrosurgical device of claim 20, wherein said return electrode is coupled to said outer assembly.

22. A method for incrementally vaporizing a mass of tissue at a target site in the body of a patient, said method comprising the step of:
a. introducing the electrosurgical device of claim 1 into the patient, using the sharp distal to penetrate said tissue mass, and manipulating the device such that said distal opening is positioned within said tissue mass, proximate to the target tissue;
b. rotating said proximal housing about the longitudinal axis of said shaft so as to cause a distal portion of said active electrode to pass through said distal opening and radially project into said target tissue;
c. applying a high-frequency voltage sufficient to provide arcing and vaporization of said target tissue to said distal portion of said active electrode;
d. optionally retracting said active electrode and repositioning the electrosurgical device and repeating steps (b) and (c) as needed to achieve complete vaporization and removal of the mass of tissue at the target site; and
e. optionally applying a voltage to said active electrode that is insufficient to provide arcing and vaporization but can sufficiently heat tissue adjacent to the target site so as to thermally treat, coagulate, and/or cauterize said tissue.

23. The method of claim 22, wherein said target tissue is a tumor.

24. The method of claim 22, wherein the sharpened distal tip of said outer assembly penetrates an exterior layer of said tissue mass and extends through the tumor tissue.

25. The method of claim 22, wherein said high-frequency voltage comprises RF energy.

26. The method of claim 22, wherein said introduction step (a) is monitored with the use of an external imaging system.

27. The method of claim 26, wherein said imaging system is selected from the group consisting of MRI, CT, PET, ultrasonic, x-ray, thermographic, photo-acoustic, gamma camera, and fluoroscopic systems.

28. The method of claim 22, wherein the sufficiency of the vaporization of said target tissue is determined by means of an external imaging system.

29. The method of claim 22, further comprising the step of positioning a return electrode proximate to the distal portion of said active electrode so as to concentrate current flow between the active and return electrodes, through the tumor.

30. The method of claim 29, wherein said return electrode is positioned at an accessible site on the patient's body.

31. The method of claim 29, wherein said return electrode is positioned on said electrosurgical device.

32. The method of claim 22, further the step of supplying an irrigant to the target site.

33. The electrosurgical device of claim 32, wherein said irrigant is selected from a liquid, a gas or a combination thereof.

34. The method of claim 22, further the step of aspirating vaporization by-products from the target site.
